(19) United States
(12) Patent Application Publication
(10) Pub. No.: US 2019/0302130 A1
(43) Pub. Date: Oct. 3, 2019

(54) HAEMOGLOBIN TEST KIT

(71) Applicant: Cell ID Pte Ltd, Singapore (SG)

(72) Inventor: Lye Hock SIM, Singapore (SG)

(21) Appl. No.: 16/315,877

(22) PCT Filed: Jul. 7, 2016
(86) PCT No.: PCT/SG2016/050314

§ 371 Date: Jan. 7, 2019

(51) Int. Cl. G01N 33/72; G01N 33/558; G01N 33/543

(52) U.S. Cl. CPC G01N 33/726; G01N 33/54386; G01N 33/558

(57) ABSTRACT

A haemoglobin test kit comprising: a lateral flow test strip having a sample collection portion, a conjugate portion and a plurality of test lines downstream of the conjugate portion, the conjugate portion comprising haemoglobin antibody conjugated with coloured particles to bind with haemoglobin in a sample fluid; wherein each test line comprises a different concentration of haemoglobin antibody, and wherein each test line comprises a particular concentration of haemoglobin antibody tuned to bind with haemoglobin in the sample fluid.

### FIELD OF THE INVENTION

This invention relates to a haemoglobin test kit.

### BACKGROUND OF THE INVENTION

In diabetes management, as stated in “Use of Glycated Haemoglobin (HbA1c) in the Diagnosis of Diabetes Mellitus”—an Abbreviated Report of a WHO (World Health Organization) Consultation, “HbA1c reflects average plasma glucose over the previous eight to 12 weeks. It can be performed at any time of the day and does not require any special preparation such as fasting. These properties have made it the preferred test for assessing glycaemic control in people with diabetes. More recently, there has been substantial interest in using it as a diagnostic test for diabetes and as a screening test for persons at high risk of diabetes. Owing in large part to the inconvenience of measuring fasting plasma glucose levels or performing an OGTT (oral glucose tolerance test), and day-to-day variability in glucose, an alternative to glucose measurements for the diagnosis of diabetes has long been sought. HbA1c has now been recommended by an International Committee and by the ADA (American Diabetes Association) as a means to diagnose diabetes. Although it gives equal or almost equal sensitivity and specificity to a fasting or post-load glucose measurement as a predictor of prevalent retinopathy, it is not available in many parts of the world. Also, many people identified as having diabetes based on HbA1c will not have diabetes by direct glucose measurement.

“An HbA1c of 6.5% is recommended as the cut point for diagnosing diabetes. A value of less than 6.5% does not exclude diabetes diagnosed using glucose tests. and vice versa.

“The use of HbA1c can avoid the problem of day-to-day variability of glucose values, and importantly it avoids the need for the person to fast and to have preceding dietary preparations. These advantages have implications for early identification and treatment which have been strongly advocated in recent years.

“The utility and convenience of HbA1c compared with measures of plasma glucose for the diagnosis of diabetes needs to be balanced against the fact that it is unavailable in many countries despite being a recognized valuable tool in diabetes management.”

As stated above, despite the advantages in using HbA1c, testing for HbA1c is unavailable in many parts of the world because it is unaffordable in most low and middle-income countries. The presently available quantitative HbA1c tests require obtaining a blood sample, lysing red blood cells in the sample with a lysis buffer to release the haemoglobin in the red blood cells, placing the lysed sample in a test cartridge and inserting the test cartridge into an correctly calibrated machine or analyser that determines the percentage of HbA1c in the total haemoglobin (Hb). Appreciably, this requires the test to be done in a laboratory or clinic with a stable power supply, even if the blood sample may be obtained elsewhere. Unfortunately, in many parts of the world, it is prohibitively costly to set up such laboratories or clinics, resulting in HbA1c not being available as a means of diagnosing and managing diabetes in the local population.

Although there has been an attempt to simplify the HbA1c test by providing a HbA1c test kit that contains a test strip with two lines that become coloured to indicate the test result, in this test, HbA1c value is determined by the user visually comparing the coloration of the lines. If the HbA1c line is almost the same colour as the Hb line, the results are normal. If the HbA1c line is darker than the Hb line, diabetes is suspected. Unfortunately, this does not provide any actual quantitative value of HbA1c and is subject to the user's personal interpretation of how much the HbA1c line is perceived to be darker than the Hb line.

From the above, it can be seen that there is a need for an affordable and feasible way to test for HbA1c that gives a reliable quantitative result, in order to make available the use of HbA1c for patients all over the world.

### SUMMARY OF INVENTION

According to a first aspect, there is provided a haemoglobin test kit comprising: a lateral flow test strip having a sample collection portion, a conjugate portion and a plurality of test lines downstream of the conjugate portion, the conjugate portion comprising haemoglobin antibody conjugated with coloured particles to bind with haemoglobin in a sample fluid; wherein each test line comprises a different concentration of haemoglobin antibody, and wherein each test line comprises a particular concentration of haemoglobin antibody tuned to bind with haemoglobin in the sample fluid.

Concentration of haemoglobin antibody in the plurality of test lines may increase for successive test lines in a downstream direction.

A most downstream visible test line may indicate percentage of haemoglobin contained in the sample fluid.

The haemoglobin test kit may further comprise visible markings that correspond with locations of the test lines in the test strip, the visible markings each indicating a particular percentage of haemoglobin that may be contained in the sample fluid.

The haemoglobin test kit may further comprise a further set of markings indicating blood glucose levels that correspond to specific percentages of haemoglobin in the visible markings.

The haemoglobin may be glycated haemoglobin.

### BRIEF DESCRIPTION OF FIGURES

In order that the invention may be fully understood and readily put into practical effect there shall now be described by way of non-limitative example only exemplary embodiments of the present invention, the description being with reference to the accompanying illustrative drawings.

FIG. 1 is a top view of an exemplary embodiment of a haemoglobin test kit.

FIG. 2 is an exploded assembly illustration of the haemoglobin test kit of FIG. 1 before use.

### DETAILED DESCRIPTION

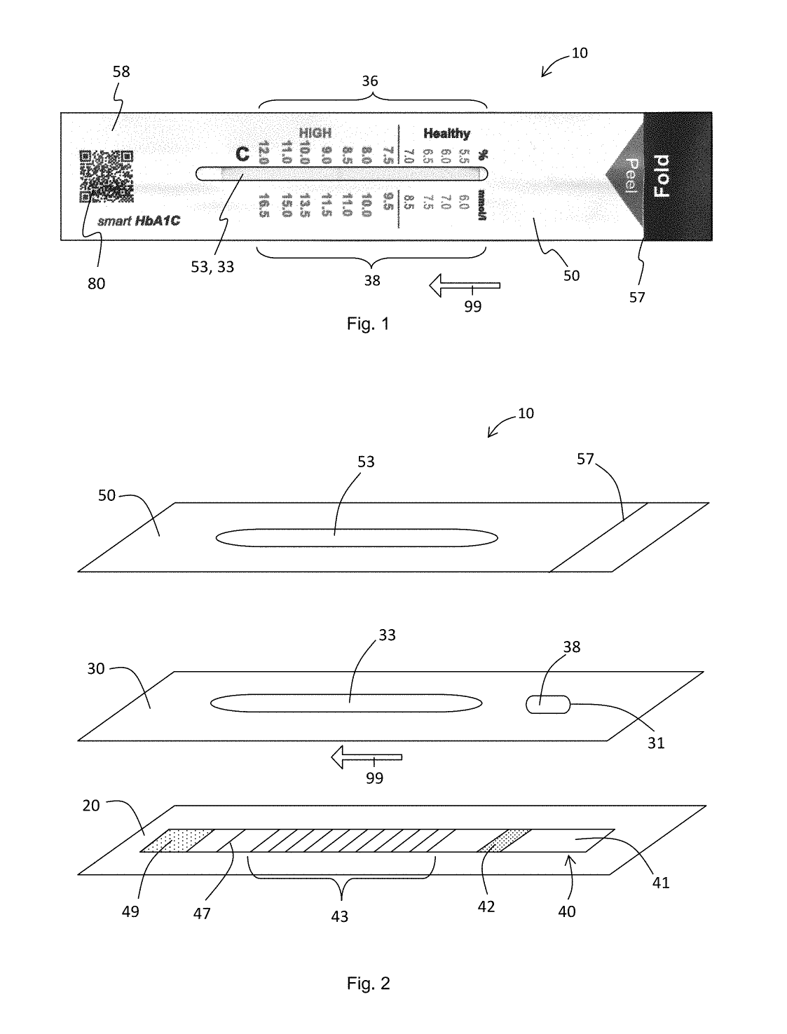

Exemplary embodiments of the haemoglobin test kit 10 will be described below with reference to FIGS. 1 and 2.

As shown in FIGS. 1 and 2, the haemoglobin test kit 10 is a lateral flow test kit comprising a lateral flow test strip 40. The test strip 40 has a sample collection portion 41, a conjugate portion 42 and a plurality of test lines 43 downstream of the conjugate portion 42. The test strip 40 preferably also has at least one control line 47 downstream of the plurality of test lines 43, and an absorbent pad 49 at the downstream end of the test strip 40. The downstream direction is indicated by arrow 99 in FIGS. 1 and 2. It should be noted that in FIG. 2 where the lateral flow test strip 40 can be seen, the test lines 43 and control lines 47 are rendered visible only for illustration purposes. In reality, the test lines 43 and control lines 47 are invisible before use of the haemoglobin test kit 10.

As shown in FIG. 1, the haemoglobin test kit 10 is provided with visible markings 36 that correspond with locations of the test lines 43 in the test strip 40. The markings 36 each indicate a particular percentage of haemoglobin that may be in the sample fluid. Preferably, the haemoglobin test kit 10 also comprises a further set of markings 38 to indicate blood glucose levels (in mmol/l) that correspond with specific percentages of haemoglobin.

In the test strip 40, each test line 43 comprises a different concentration of haemoglobin antibody. The haemoglobin antibody may be an antibody for haemoglobin (Hb) or glycated haemoglobin (HbA1c), according to which type of haemoglobin the test kit is intended to be used for. Each test line comprises a particular concentration of haemoglobin antibody that is tuned to bind with haemoglobin in a sample fluid that flows through the test strip 40. Before binding with the haemoglobin antibody in a test line, haemoglobin in the sample fluid is bound to a conjugate that becomes visible at a test line when a sufficient quantity of haemoglobin in the sample fluid has been immobilized at that test line.

The conjugate is provided on the test strip 40 in a conjugate portion 42 adjacent the sample collection portion 41. The conjugate may comprise any known coloured particles such as colloidal gold nanoparticles or latex microspheres conjugated with haemoglobin antibody. haemoglobin in the sample fluid thus binds with the haemoglobin antibody in the conjugate to pick up the coloured particles, forming a complex comprising haemoglobin conjugated with coloured particles. As the complex flows across the test lines 43, haemoglobin in the complex also binds with the haemoglobin antibody at the test lines to immobilize the coloured particles at the test lines. In this way, a particular test line becomes visible only when a sample fluid that flows across that particular test line 43 contains at least a same percentage of haemoglobin as the particular percentage of haemoglobin for which the particular test line 43 is tuned to bind with and indicate, as a result of a sufficient concentration of coloured particles being immobilized at the particular test line together with the haemoglobin in the complex.

The concentration of haemoglobin antibody comprised in each test line 43 increases for successive test lines 43 in the downstream direction 99. Thus, referring to FIG. 1, the test line at the 6.0% marking comprises a higher concentration of haemoglobin antibody than the test line at the 5.5% marking. In this way, when a sample fluid is placed on the sample collection portion 41 and flows across the test lines 43, if the sample fluid contains haemoglobin, the percentage of haemoglobin in the sample fluid may be quantitatively known by observing which of the test lines 43 become visible in a reaction window 33 of the haemoglobin test kit 10. Among the test lines that become visible, the most downstream visible test line indicates the actual percentage of haemoglobin in the sample fluid.

For example, referring to FIG. 1, when a sample fluid containing 7.0% haemoglobin is tested using the haemoglobin test kit 10, test lines 43 at the 5.5%, 6.0%, 6.5%, and 7.0% markings will become visible. This is because the 7.0% haemoglobin present in the sample fluid is sufficient to bind with the concentration of haemoglobin antibody comprised by the test lines at the 5.5%, 6.0%, 6.5%, and 7.0% markings to cause these test lines to become visible. The remaining test lines that indicate a haemoglobin percentage greater than 7.0% stay invisible because the 7.0% haemoglobin present in the sample fluid is insufficient to bind with the remaining test lines to cause the remaining test lines to become visible. This is because the remaining test lines comprise higher concentrations of haemoglobin antibody that are tuned to bind with and indicate higher percentages of haemoglobin than 7.0%. In this example, the 7.0% line being the most downstream of the visible test lines thus indicates that the sample fluid has 7.0% haemoglobin.

Similarly, if another sample fluid is tested with the haemoglobin test kit 10 and the test lines at the 5.5%, 6.0%, 6.5%, 7.0%, 7.5%, 8.0%, and 8.5% markings become visible while the remaining test lines 43 stay invisible, it can be concluded that the sample fluid contains 8.5% haemoglobin.

The control line 47 comprises antibody for binding with the haemoglobin antibody in the conjugate to indicate that the test is valid when the control line 47 becomes visible.

The sample fluid typically comprises a mixture of a patient's blood sample and a commercially available lysis buffer. In use, the sample fluid may be prepared by lancing a patient's fingertip to obtain a drop of blood, placing the drop of blood in about 0.5 ml of the lysis buffer, and agitating the mixture of the drop of blood and the lysis buffer for about 5 seconds to obtain the sample fluid.

In the exemplary embodiment shown in FIGS. 1 and 2, the haemoglobin test kit 10 comprises a base sheet 20, a sealing sheet 30 having a sealed sample collection 31 opening before use of the haemoglobin test kit 10, the lateral flow test strip 40 sealed between the base sheet 20 and the sealing sheet 30 before use of the haemoglobin test kit 10, and a top sheet 50. The sealing sheet 30 is provided with a transparent portion configured as a reaction window 33 aligned over the test lines 43 and the control line 47 so that they 43, 47, can be seen through the reaction window 33. The top sheet 50 is configured to be attached to the sealing sheet 30 before use of the haemoglobin test kit 10 to keep the sample collection opening 31 sealed. As the top sheet 50 is layered over all of the sealing sheet 30, the top sheet 50 also comprises a transparent portion 53 aligned with the reaction window 33 of the sealing sheet 30 to allow the test lines 43 and the control line 47 to be seen through both the sealing sheet 30 and the top sheet 50 layers.

In an alternative embodiment, the top sheet 50 may be layered only partially over the sealing sheet 30 without being layered over the reaction window 33, in which case no transparent portion needs to be provided in the top sheet 50.

The base sheet 20, sealing sheet 30 and top sheet 50 are preferably rectilinear in shape and of the same size so that the haemoglobin test kit 10 is a simple, almost flat rectangular package.

As shown in FIG. 2, the top sheet 50 is further configured to be at least partially detached from the sealing sheet 30 to open the sample collection opening 31 during use of the haemoglobin test kit 10. When the sample collection opening 31 has been opened, the sample collection portion 41 of the test strip 40 is exposed through the sample collection opening 31 and can then receive a fluid analyte thereon. This may be achieved by layering the top sheet 50 over the sealing sheet 30 and providing the top sheet 50 with appropriately located die cut lines 57 (e.g. on at least one side of the sample collection opening 31) to allow the top sheet 50 to be at least partially peeled away from the sealing sheet 30.

The sample collection opening 31 is preferably sealed by a cover 38 before use of the haemoglobin test kit 10. The cover 38 is attached to the top sheet 50 such that at least partially detaching the top sheet 50 from the sealing sheet 30 detaches the cover 38 from the sealing sheet 30 to open the sample collection opening 31. The cover 38 is preferably integral with or part of the sealing sheet 30 before use of the haemoglobin test kit 10.

The top sheet 50 is preferably also configured to be re-attachable to the sealing sheet 30 to cover the sample collection opening 31 after use of the haemoglobin test kit 10, in order to prevent the sample fluid on the sample collection portion 41 from coming into contact with another object. This is a safety or hygiene feature to minimize or prevent human handlers of the used haemoglobin test kit 10 from being contaminated by contaminants in the sample fluid, and to minimize or prevent cross contamination with other used haemoglobin test kits 10.

The base sheet 20, sealing sheet 30 and at least one top sheet 50 each preferably comprise a moisture barrier polymeric film so that the at least one test strip 40 is kept well sealed between the base sheet 20 and sealing sheet 30 before use of the haemoglobin test kit 10.

In this way, the haemoglobin test kit 10 requires no additional moisture barrier packaging to keep the at least one test strip 40 stable during storage and transportation of the haemoglobin test kit 10 before use. This also greatly reduces the size of the haemoglobin test kit 10 and the space it takes up, which would have a significant impact particularly when haemoglobin test kits need to be moved in areas with poor transportation networks or accessibility. For example, a single healthcare worker going on foot to a remote location will be able to take with him or her a great many more of the haemoglobin test kits 10 of the present invention in a single hand-carry bag or case or even clothing pocket as compared to existing haemoglobin test kits with plastic cassettes in foil bags that would be significantly more bulky to carry. Doing away with the need for an additional moisture barrier foil bag and plastic cassette also significantly reduces the cost of the haemoglobin test kit 10 of the present invention, since each foil bag and each plastic cassette contributes to the total cost of each traditionally available haemoglobin test kit.

In addition, the haemoglobin test kit 10 is preferably provided with at least one quick response (QR) code 80 on the haemoglobin test kit 10, more preferably located on the top sheet 50 for easy access. The at least one QR code 80 allows information such as manufacturing date, expiry date and source information of the at least one test strip 40 and the haemoglobin test kit 10 itself to be stored and retrieved, as well as allowing the haemoglobin test kit 10 to be associated or tagged with a single specific source of the fluid analyte. The specific source may be a patient or any other sample fluid, depending on the usage application of the haemoglobin test kit 10. The QR code is preferably located on a part 58 of the top sheet 50 where the part 58 of the top sheet 50 is never detached from the sealing sheet 30, or on a part of the sealing sheet 30 that is not overlayed by a top sheet 50 (depending on the configuration of the haemoglobin test kit 10). In this way, the quick response code 80 is never separated from the test strip 40 in the haemoglobin test kit 10 after use, and each test strip 40 can be correctly traced to its specific sample fluid source. Having the quick response code 80 on each haemoglobin test kit 10 allows each haemoglobin test kit 10 to be indelibly and indubitably associated with only one specific sample fluid source, thereby minimizing or preventing mix-ups in test results from occurring.

The haemoglobin test kit 10 is preferably provided or sold together with the lysis buffer to allow diabetes patients to prepare the sample fluid and test for haemoglobin using the haemoglobin test kit 10 at home. In this way, the present invention provides a low cost, low bulk haemoglobin test kit 10 that can be used at the point of care and associated with a specific sample fluid source (e.g. a specific patient) without requiring a costly laboratory or clinic set-up to run the tests for haemoglobin.

Whilst there has been described in the foregoing description exemplary embodiments of the present invention, it will be understood by those skilled in the technology concerned that many variations in details of design, construction and/or operation may be made without departing from the present invention. For example, while it has been described that the base sheet, sealing sheet and top sheet are preferably made of a moisture barrier polymeric film, they may alternatively be made of appropriately laminated paper with sufficient moisture barrier properties required for stable storage of the test strips therein. It is further envisaged that in various embodiments of the haemoglobin test kit, the top sheet may or may not be layered over all of the sealing sheet so long as it is layered over the sample collection opening to keep the sample collection opening sealed before use of the haemoglobin test kit. In addition to the above described examples and alternative configurations, any other possible configurations of lateral flow test kits may be used to house the test strip 40 so long as the test strip comprises the sample collection portion and the plurality of test lines as described above.

1. A haemoglobin test kit comprising:
a lateral flow test strip having a sample collection portion, a conjugate portion and a plurality of test lines downstream of the conjugate portion, the conjugate portion comprising haemoglobin antibody conjugated with coloured particles to bind with haemoglobin in a sample fluid;
wherein each test line comprises a different concentration of haemoglobin antibody, and wherein each test line comprises a particular concentration of haemoglobin antibody tuned to bind with haemoglobin in the sample fluid.

2. The haemoglobin test kit of claim 1, wherein concentration of haemoglobin antibody in the plurality of test lines increases for successive test lines in a downstream direction.

3. The haemoglobin test kit of claim 1, wherein a most downstream visible test line indicates percentage of haemoglobin contained in the sample fluid.

4. The haemoglobin test kit of claim 1, further comprising visible markings that correspond with locations of the test lines in the test strip, the visible markings each indicating a particular percentage of haemoglobin that may be contained in the sample fluid.

5. The haemoglobin test kit of claim 1, further comprising a further set of markings indicating blood glucose levels that correspond to specific percentages of haemoglobin in the visible markings.

6. The haemoglobin test kit of claim 1, wherein the haemoglobin is glycated haemoglobin.
